(19) United States
(12) Patent Application Publication
(10) Pub. No.: US 2026/0351384 A1
(43) Pub. Date: Nov. 5, 2026

(54) SUPPORT SYSTEM, SUPPORT METHOD, AND INFORMATION PROCESSING APPARATUS

(71) Applicant: SONY GROUP CORPORATION, TOKYO (JP)

(72) Inventors: MASAHIRO MIURA, TOKYO (JP); TAKU TANAKA, TOKYO (JP); TAKUYA KISHIMOTO, TOKYO (JP); KENJI YAMANE, TOKYO (JP); RITSUKO KANO, TOKYO (JP); SAKIKO YASUKAWA, TOKYO (JP); AYUMU TAGUCHI, TOKYO (JP)

(21) Appl. No.: 19/486,044

(22) PCT Filed: May 13, 2024
(86) PCT No.: PCT/JP2024/017555

§ 371 Date: Nov. 20, 2025

(30) Foreign Application Priority Data
Jun. 5, 2023 (JP) 2023-092587

(51) Int. Cl. G16H 20/70

(52) U.S. Cl. CPC G16H 20/70

(57) ABSTRACT

A support system includes an acquisition unit configured to acquire user information of a user, a model generation unit configured to generate a user model of the user based on the user information, an information generation unit configured to generate, based on the user model, support information supporting setting of a sense of values and an action goal of the user, and a presentation unit configured to present the support information to the user.

### TECHNICAL FIELD

The present disclosure relates to a support system, a support method, and an information processing apparatus.

### BACKGROUND ART

For example, PTL 1 discloses a conversation generation technology for encouraging a user to perform an action for achieving a received goal of the user.

### CITATION LIST

### Patent Literature

PTL 1: JP 2017-73125 A

### SUMMARY

### Technical Problem

In the field of psychotherapy, there is known a therapy for improving mental distress of a patient by setting a sense of values of the patient and an action goal aligned with the sense of values. A technology for supporting such a therapy is not specifically studied in PTL 1.

One aspect of the present disclosure is to support the setting of a sense of values and an action goal.

### Solution to Problem

A support system according to an aspect of the present disclosure includes an acquisition unit configured to acquire user information of a user, a model generation unit configured to generate a user model of the user based on the user information, an information generation unit configured to generate, based on the user model, support information supporting setting of a sense of values and an action goal of the user, and a presentation unit configured to present the support information to the user.

A support method according to an aspect of the present disclosure includes acquiring user information of a user, generating a user model of the user based on the user information, generating, based on the user model, support information supporting setting of a sense of values and an action goal of the user, and presenting the support information to the user.

An information processing apparatus according to an aspect of the present disclosure includes a model generation unit configured to generate, based on user information of a user, a user model of the user, and an information generation unit configured to generate, based on the user model, support information supporting setting of a sense of values and an action goal of the user.

### BRIEF DESCRIPTION OF DRAWINGS

FIG. 1 is a diagram illustrating an example of a schematic configuration of a support system 1 according to an embodiment.

FIG. 2 is a diagram illustrating an example of user information 30.

FIG. 3 is a diagram illustrating an example of a user model 80.

FIG. 4 is a diagram illustrating specific examples of the user information 30 and the user model 80 after conversion.

FIG. 5 is a diagram illustrating specific examples of the user information 30 and the user model 80 after conversion.

FIG. 6 is a diagram illustrating an example of support information 91.

FIG. 7 is a diagram illustrating a specific example of generation of the support information 91 based on the user model 80.

FIG. 8 is a flowchart illustrating an example of processing (support method) executed in the support system 1.

FIG. 9 is a flowchart illustrating an example of the processing (support method) executed in the support system 1.

FIG. 10 is a flowchart illustrating an example of the processing (support method) executed in the support system 1.

FIG. 11 is a flowchart illustrating an example of the processing (support method) executed in the support system 1.

FIG. 12 is a flowchart illustrating an example of the processing (support method) executed in the support system 1.

FIG. 13 is a diagram illustrating a modified example.

FIG. 14 is a diagram illustrating an example of a hardware configuration of an apparatus or the like.

### DESCRIPTION OF EMBODIMENTS

Hereinafter, embodiments of the present disclosure will be described in detail with reference to the drawings. In each of the following embodiments, the same elements are denoted by the same reference signs, and redundant description thereof will be omitted.

The present disclosure will be described in the order of the following items.
0. Introduction
1. Embodiment
2. Modified Example
3. Example of Hardware Configuration
4. Summary

### 0. Introduction

In cognitive behavioral therapy, a therapy has been established in which a counselor has a dialogue with a patient and has the patient set, implement, and, if necessary, correct a sense of values that the patient places emphasis on in their life and an action goal aligned with the sense of values, thereby improving mental distress of the patient. An example of such a therapy is a behavioral activation therapy.

It is expected to develop a technology for supporting the setting or the like of a sense of values and an action goal, for example, a digital application technology for simplifying the setting or the like. However, there has been no technology for extracting information from a dialogue with a patient like a counselor and setting a sense of values and an action goal according to the features of the patient (by individualizing a response for each patient).

One of the problems is the amount of information. In order to acquire (collect) a lot of information related to a patient (hereinafter, also referred to as a “user”) and achieve sufficiently individualized interaction, it is necessary to input a lot of information. That is, in order to achieve individualization for each user, it is necessary to acquire a certain amount of information so that the information serves as a reference of grouping for distinguishing the user from other users. However, when the user is forced to input information to an application for this acquisition, the burden on the user increases.

According to the disclosed technology, information consciously (proactively) input by a user and information automatically acquired without being consciously input by the user are combined, and thus information on the user is acquired in a manner less burdensome for the user. As will be described in detail below, a user model representing the personality of the user is constructed from the acquired information on the user. The user model is used to support the user in the setting or the like of a sense of values and an action goal of the user. As in counseling by a counselor, it is possible to conduct an individualized therapy on the user to improve the therapeutic experience of the user.

### 1. Embodiment

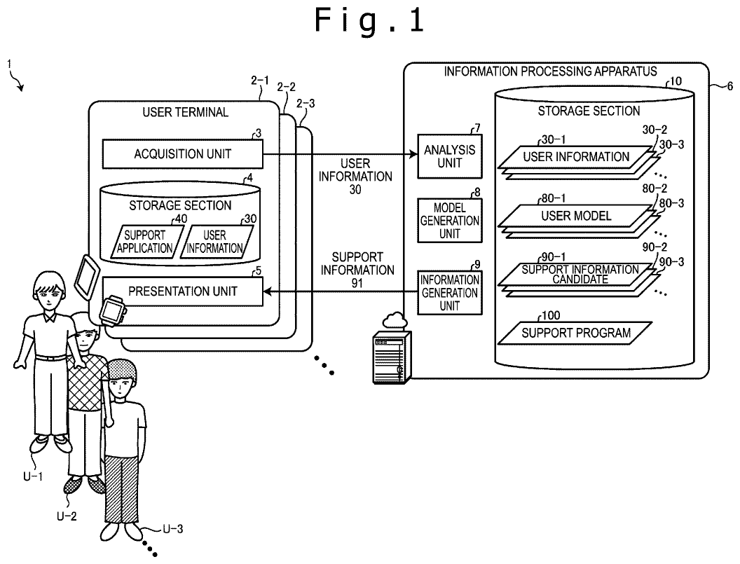

FIG. 1 is a diagram illustrating an example of a schematic configuration of a support system 1 according to an embodiment. A user of the support system 1 is referred to as a user U. The user U is, for example, a patient of psychotherapy. An example of the psychotherapy is a cognitive behavioral therapy, and more specifically, a behavioral activation therapy, acceptance & commitment therapy, or the like. While not limited thereto, a patient undergoing any therapy including the setting or the like of a sense of values and an action goal of the user U can be the user U. The patient in the present disclosure is not limited to a patient in a strict sense, and may be understood to include, for example, a person with mental distress, a person aiming to improve well-being, and the like.

There may be a plurality of users U, and FIG. 1 illustrates three users U. In order to distinguish the users U from each other, the users U are referred to as a user U-1, a user U-2, a user U-3, and the like and illustrated. When these users are not particularly distinguished from each other, each user is simply referred to as a user U.

The support system 1 supports a therapy for the user U. For example, the setting of a sense of values that the user U places importance on in their life (which can also be said to be clarification of the sense of values) is supported based on information including an activity record and the like of the user U. Further, the setting of an action goal aligned with the sense of values and the execution of an action related to the action goal are supported. The action is executed and then looked back, and thus the correction of the setting content is also supported.

The support system 1 includes a user terminal 2 and an information processing apparatus 6. The user terminal 2 and the information processing apparatus 6 communicate with each other via a communication network such as the Internet.

The user terminal 2 is used to acquire information related to the user U and present (display or the like) support information 91 described below to the user U. Examples of the user terminal 2 include a smartphone, a tablet terminal, a PC, a wearable device (such as a wearable watch), and the like, and in FIG. 1, the user terminal 2 is illustrated as an apparatus including a smartphone and a wearable device. The wearable device acquires biological information (vital data) of the user U.

There may be a plurality of user terminals 2 corresponding to the plurality of users U. The user terminal of the user U-1 is referred to as a user terminal 2-1 and illustrated. The user terminal of the user U-2 is referred to as a user terminal 2-2 and illustrated. The user terminal of the user U-3 is referred to as a user terminal 2-3 and illustrated. When these user terminals are not particularly distinguished from each other, each user terminal is simply referred to as a user terminal 2.

The user terminal 2 includes an acquisition unit 3, a storage section 4, and a presentation unit 5. The storage section 4 will be described first, and then the acquisition unit 3 and the presentation unit 5 will be described.

The storage section 4 stores information used in the user terminal 2. Examples of the information stored in the storage section 4 include user information 30 and a support application 40. The user information 30 will be described below. The support application 40 is an application (software, application program) for causing a computer to function as the user terminal 2. The support application 40 operates on the user terminal 2 and supports the setting or the like of a sense of values and an action goal of the user U. For example, the support application 40 operates so as to obtain log information (an example of the user information 30) of the support application 40 described below. The user information 30 will be described below.

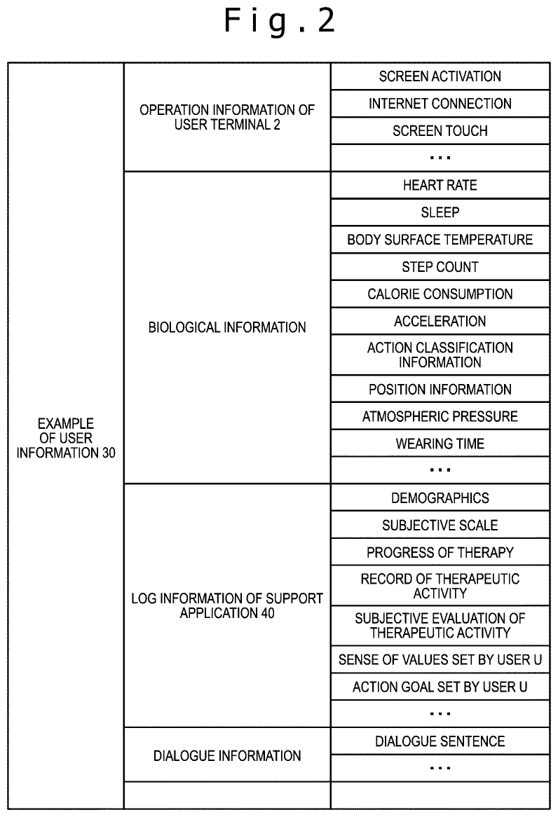

The acquisition unit 3 acquires (collects) information related to the user U. The information related to the user U is referred to as user information 30. The acquisition of the user information 30 by the acquisition unit 3 may be performed by the user U consciously operating the user terminal 2 to input the information, or may be automatically performed while the user U is unaware. Any type of information that can be obtained by the user terminal 2 may be acquired as the user information 30. The description will be made with reference to FIG. 2.

FIG. 2 is a diagram illustrating an example of the user information 30. Examples of the user information 30 include operation information of the user terminal 2, biological information, log information of the support application 40, and dialogue information.

The operation information of the user terminal 2 includes operation information of the user terminal 2 other than the log information of the support application 40. For example, an operation log or information obtained therefrom can be the operation information of the user terminal 2. Examples of such information include screen activation, Internet connection, screen touch, and the like. For example, information such as the number of times, the frequency, and the time-series state transition thereof can be the operation information of the user terminal 2.

The biological information is biological information of the user U. Examples of the biological information include heart rate, sleep, body surface temperature, step count, calorie consumption, acceleration, action classification information, position information, atmospheric pressure, and wearing time. The acceleration indicates the acceleration of a portion of the user U where the wearable device is worn. Examples of the action classification information include running, swimming, cycling, traveling by train, and the like. The position information is acquired by a GPS sensor or the like. Note that various types of biological information other than those illustrated in FIG. 2 may also be included in the user information 30. Other examples of the biological information include electrocardiogram, blood flow, vital capacity, myoelectric potential, blood glucose level, weight, vibration, impact, perspiration, and the like.

The log information of the support application 40 is log information of the support application 40 other than dialogue. Examples of the log information of the support application 40 include demographics, a subjective scale, progress of a therapy, a record of a therapeutic activity, a subjective evaluation of the therapeutic activity, a sense of values set by the user U, and an action goal set by the user U. Examples of the demographic information include age, gender, occupation, family, medical history, and the like. The subjective scale information is subjective scale information indicating the mental state and personality of the user U, and is acquired using, for example, PHQ-9, GAD-7, 16Personalities, or the like.

The dialogue information is information on a dialogue performed by the user U using the user terminal 2, and examples thereof include a dialogue sentence input by the user U in a dialogue with the support system 1. The dialogue may be performed via the support application 40 or may be performed using a function on the user terminal 2 other than the support application 40. Unless otherwise specified, the dialogue is performed via the support application 40.

Among the above-described pieces of information, the operation information of the user terminal 2, the biological information, and the log information of the support application 40 can be obtained without being consciously input by the user U. The dialogue information is obtained by being consciously input by the user U. It is not necessary to acquire all of the above pieces of information as the user information 30. Information that can be acquired within the range of the design or the like of the user terminal 2 can be the user information 30.

Referring back to FIG. 1, the user information 30 acquired by the acquisition unit 3 is stored in the storage section 4 and transmitted from the user terminal 2 to the information processing apparatus 6. Although details will be described below, the information processing apparatus 6 generates, based on the user information 30 from the user terminal 2, support information 91 for supporting the setting or the like of valuing and an action goal of the user U. The support information 91 is transmitted from the information processing apparatus 6 to the user terminal 2.

The presentation unit 5 of the user terminal 2 presents the support information 91 from the information processing apparatus 6 to the user U. The presentation mode of the support information 91 is not particularly limited, but for example, a dialogue sentence is displayed, or various notifications are performed. The notifications may be performed via the support application 40 or may be performed via a service other than the support application 40 (for example, an external mailer or the like). Further details of the support information 91 will be described below.

The information processing apparatus 6 includes an analysis unit 7, a model generation unit 8, an information generation unit 9, and a storage section 10. The storage section 10 will be described first, and then the analysis unit 7, the model generation unit 8, and the information generation unit 9 will be described.

The storage section 10 stores information used by the information processing apparatus 6. Examples of the information stored in the storage section 10 include user information 30, a user model 80, a support information candidate 90, and a support program 100. The storage section 10 can also be referred to as a database (DB) that stores the user information 30, the user model 80, and the support information candidate 90. The support program 100 is a program (software, application program) for causing a computer to function as the information processing apparatus 6. The user model 80 and the support information candidate 90 as well as the user information 30 will be described in detail below.

The analysis unit 7 analyzes the user information 30 from the user terminal 2. For example, the analysis unit 7 converts the user information 30 into a format that can be stored as a DB, more specifically, a format suitable for use in the model generation unit 8 described below, and stores the user information 30 in the storage section 10.

There can be a plurality of pieces of user information 30 corresponding to the plurality of users U. The analysis unit 7 stores these pieces of user information 30 in the storage section 10 in a distinguishable manner for each user U. The user information of the user U-1 is referred to as user information 30-1 and illustrated. The user information of the user U-2 is referred to as user information 30-2 and illustrated. The user information of the user U-3 is referred to as user information 30-3 and illustrated. When these pieces of user information are not particularly distinguished from each other, each piece of user information is simply referred to as user information 30.

At least part of information in the user information 30 may be transmitted from the analysis unit 7 to the information generation unit 9. Examples of such user information 30 include the above-described dialogue information (dialogue sentence or the like).

The model generation unit 8 generates, based on the user information 30, a model of the user U, for example, a model of the user U expressing the personality of the user U. Such a model is referred to as a user model 80. The user model 80 includes various feature amounts obtained from the user information 30.

The model generation unit 8 acquires the user information 30 from the storage section 10 and, for example, converts the acquired information to obtain (extract, calculate, or the like) the feature amounts of the user U. The model generation unit 8 generates the user model 80 based on the obtained feature amounts.

In the user model 80, various feature amounts of the user U may be described as a vector. The vector may be obtained by dimensionally reducing all variables of the feature amounts. The dimension reduction means that the dimension of the original information is reduced by one or more dimensions. Examples of dimension reduction methods include PCA, t-SNE, UMAP, and the like.

The generated user model 80 is a model that expresses the personality of the user U, and is a model that can be, for example, discriminated or distinguished from user models 80 of the other users U.

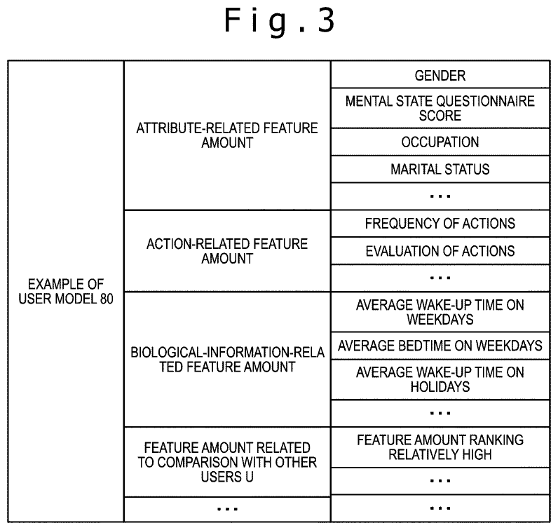

A plurality of user models 80 corresponding to the plurality of users U are generated. The user model of the user U-1 is referred to as a user model 80-1 and illustrated. The user model of the user U-2 is referred to as a user model 80-2 and illustrated. The user model of the user U-3 is referred to as a user model 80-3 and illustrated. When these user models are not particularly distinguished from each other, each user model is simply referred to as a user model 80. The user model 80 will be described with reference to FIG. 3.

FIG. 3 is a diagram illustrating an example of the user model 80. Examples of the feature amounts included in the user model 80 include an attribute-related feature amount, an action-related feature amount, a biological-information-related feature amount, and a feature amount related to comparison with the other users U.

Examples of the attribute-related feature amount include gender, mental state questionnaire score, occupation, and marital status. Examples of the mental questionnaire score include the scores of PHQ- 9, GAD- 7, 16Personalities, and the like described above. Such feature amounts can be obtained from, for example, the log information or the like of the support application 40 included in the user information 30.

Examples of the action-related feature amount include the frequency of actions and the evaluation of the actions. For example, the types of actions actually performed by the user U, the frequency of the actions, the evaluation by the user U after the actions, and the like can be the action-related feature amounts. Such feature amounts can be obtained from, for example, the log information of the support application 40 included in the user information 30, the dialogue information, and the like.

Examples of the biological-information-related feature amount include the average wake-up time on weekdays, the average bedtime on weekdays, and the average wake-up time on holidays.

Such feature amounts can be obtained from, for example, the biological information or the like included in the user information 30.

Examples of the feature amount (comparison feature amount) related to comparison with the other users U include a feature amount ranking relatively high. For example, a feature amount included in the top 10% in a user group satisfying a given criterion can be the comparison feature amount. Using not only the feature amounts related to the attribute, the action, and the biological information described above, but also the comparison feature amount increases the number of feature amounts included in the user model 80, accordingly enhances the expressiveness of the user model 80, and enables support that is more in line with the features of the user U.

For example, the comparison feature amount has a default value of 0 (false), and when a condition is satisfied, for example, when the feature amount ranks high in the user group in the above example, the value thereof is changed to 1 (true), and the comparison feature amount may be included as a feature amount of the user model 80.

A specific example of the comparison feature amount will be described. For example, when the step count and the calorie consumption of a certain user U rank high in a user group, a feature amount “TopRank10%Activity” related to the step count and the calorie consumption is added to the feature amounts of the user model 80 of the user U.

Another example of the comparison feature amount will be described. It is assumed that, for example, a topic “family” is estimated as a topic related to a certain user U from an activity record of the user U, and there are a plurality of other users U having a common topic “family”. In this case, a feature amount “TopicOfFamily” is added to the feature amounts of the user models 80 of these users U.

For example, a user model 80 including various feature amounts as described above is generated. Each feature amount may be represented by a numerical value. For example, the value indicated in the user information 30 itself may be calculated as a feature amount. A value (statistical value) obtained as summary statistics using a set of values of the user U during a certain period may be calculated as a user feature amount 80. Examples of the statistical value include the mean value, the maximum value, the minimum value, the median value, the variance, the standard deviation, and the like. Various scaling methods may be used for calculation of the feature amount. For example, the value of the feature amount may be normalized (min-max normalization) by z-score normalization or the like.

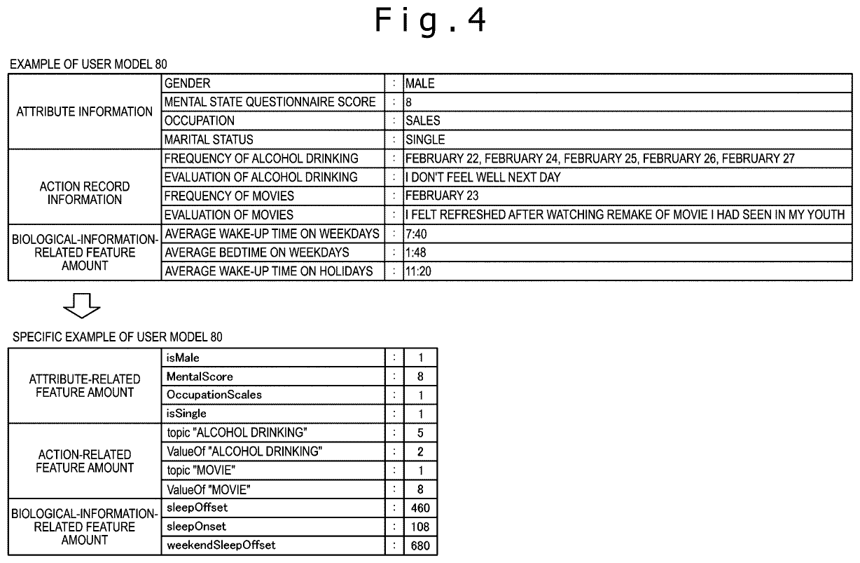

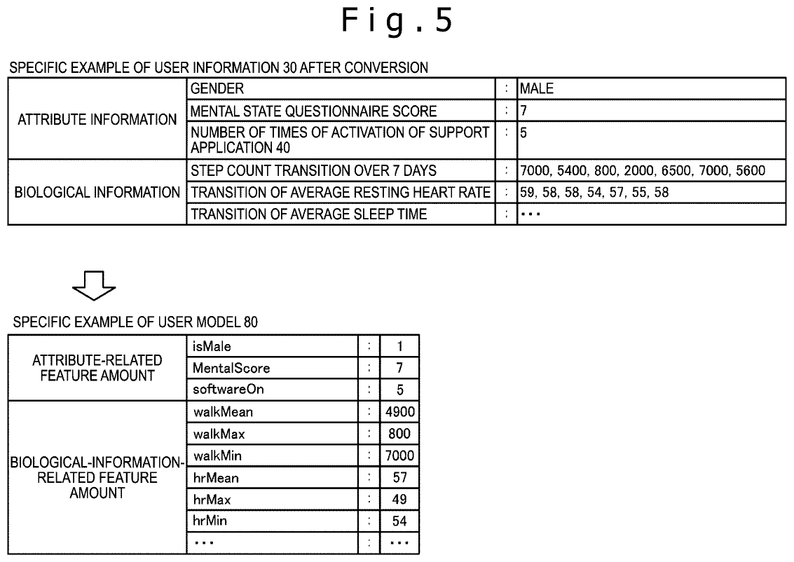

Referring back to FIG. 1, as described above, the analysis unit 7 analyzes the user information 30, converts the user information 30 into a format suitable for generation of the user model 80 by the model generation unit 8, and stores the user information 30 in the storage section 10. Specific examples of the user information 30 after the conversion and the user model 80 generated therefrom will be described with reference to FIGS. 4 and 5.

FIGS. 4 and 5 are diagrams illustrating the specific examples of the user information 30 after the conversion and the user model 80. At the upper part of FIG. 4, specific examples of attribute information, action record information, and biological information are illustrated as the user information 30 after the conversion. The attribute information and the action record information are obtained from the log information of the support application 40. The contents are as illustrated in the figure, and only some pieces of information are supplemented. The frequency of alcohol drinking and the evaluation of the alcohol drinking are described as the dates when the user U drinks alcohol, and comments (examples of dialogue sentences) input by the user U about the alcohol drinking. The frequency of movies and the evaluation of the movies are described as the dates when the user U watches the movies, and comments input by the user U about the movies. A user model 80 illustrated at the lower part of FIG. 4 is generated based on such user information 30.

In the user model 80, the attribute-related feature amounts, more specifically, the vector of the gender, the mental state questionnaire score, the occupation, and the marital status are described as isMale, MentalScore, OccupataionScales, and isSingle, together with numerical values indicating the magnitudes thereof.

In the user model 80, the action-related feature amounts, more specifically, the vector of the frequency of the alcohol drinking, the evaluation of the alcohol drinking, the frequency of the movies, and the evaluation of the movies are described as topic “alcohol drinking”, VallueOf “alcohol drinking”, topic “movie”, and ValueOf “movie”, together with numerical values indicating the magnitudes thereof.

In the user model 80, the biological-information-related feature amounts, more specifically, the vector of the average wake-up time on weekdays, the average bedtime on weekdays, and the average wake-up time on holidays are described as sleepOffset, sleepOnset, and weekendSleepOffset, together with numerical values indicating the magnitudes thereof.

At the upper part of FIG. 5, specific examples of the attribute information and the biological information are illustrated as the user information 30 after the conversion. The contents are as illustrated in the figure. A user model 80 illustrated at the lower part of FIG. 5 is generated based on such user information 30.

In the user model 80, the attribute-related feature amounts, more specifically, the vector of the gender, the mental state questionnaire score, and the number of times of activation of the support application 40 are described as isMale, MentalScore, and softwareOn, together with numerical values indicating the magnitudes thereof.

In the user model 80, the biological-information-related feature amounts, more specifically, the vector of the mean value, the maximum value, and the minimum value of the step count over seven days, the mean value, the maximum value, and the minimum value of the resting heart rate, and the like are described as walkMean, walkMax, walkMin, hrMean, hrMax, hrMin, and the like together with values indicating the magnitudes thereof.

For example, as illustrated in FIGS. 4 and 5, the user model 80 is generated based on the user information 30 after the conversion.

Referring back to FIG. 1, the user model 80 generated by the model generation unit 8 is stored in the storage section 10. When the user model 80 has already been generated and stored in the storage section 10, the user model 80 may be updated. The generation and the update of the user model 80 may be interpreted interchangeably as long as no contradiction arises.

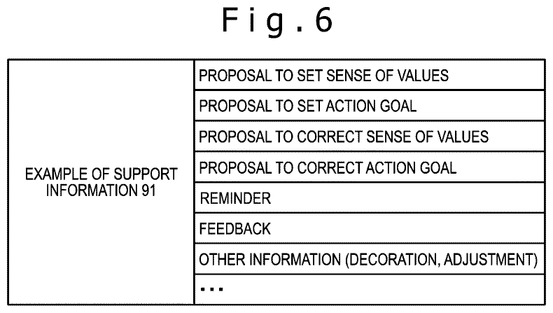

The information generation unit 9 generates support information 91 based on the user model 80. Details of the support information 91 will be described below with reference to FIG. 6. The support information 91 is transmitted from the information processing apparatus 6 to the user terminal 2.

As described above, in the user terminal 2, the presentation unit 5 presents the support information 91 from the information processing apparatus 6. The user U operates the user terminal 2 in accordance with the presented support information 91. The operation result is transmitted from the user terminal 2 to the information processing apparatus 6 as, for example, further user information 30. Further support information 91 is generated in the information processing apparatus 6 and transmitted to the user terminal 2. The user model 80 stored in the storage section 10 of the information processing apparatus 6 may be updated in response to the acquisition of the further user information 30 or at any timing.

A series of operations such as the acquisition of the user information 30, the generation or update of the user model 80, the generation and presentation of the support information 91, and the like is repeated, so that the setting or the like of a sense of values and an action goal of the user U is supported.

Since the user model 80 is generated individually for each user U, it is possible to generate and present support information 91 suitable for supporting the user U. The user U can reduce the burden of the setting or the like of the sense of values and the action on the user U, for example, the time, effort and the like required for the user U to consider the setting or the like.

A specific example of the support including the presentation of the support information 91 will be described with reference to FIG. 6.

FIG. 6 is a diagram illustrating an example of the support information 91. Examples of the support information 91 include a proposal to set a sense of values, a proposal to set an action goal, a proposal to correct the sense of values, a proposal to correct the action goal, a reminder, feedback, and other information.

The proposal to set the sense of values is a proposal for the user U to clarify and set the sense of values. For example, the proposal to set the sense of values may include presenting a list of items (domains) such as a human relationship, work, academics, mental and physical health, a hobby, volunteering, spirituality, and the like as candidates of life domains. Such a list may be prepared in advance, and is presented to the user U when the user U newly sets a sense of values, for example. The content of each item may be presented in a subdivided manner. For example, the human relationship may be subdivided into a friend relationship, a family relationship, and the like.

The presented list may be, for example, edited based on the user model 80 so as to include information that is likely to be selected by the user U. For example, among the feature amounts included in the user model 80, a list including items that are likely to be selected by the user U is presented from feature amounts derived from the occupation, family structure, activity record, and the like of the user U. Together with the list, the following sentences may be presented, for example.
“What domain do you choose as being important in your life?”
“You entered ‘pick-up and drop-off of my child’ as an activity record four times a week. Do you consider ‘family relationship’ important in your life?”
“Select any number of sentences that seem to fit your sense of values from the following sentences.”
“Select any number of images that seem to fit your sense of values from the following images.”

In response to the presentation of the list as described above, the user U selects one or more domains that the user U considers important. Note that, when there is not sufficient information for proposing candidates, for example, a plurality of images or sentences reflecting a sense of values in life (success at work, good family relationship, and the like) may be presented. Items related to the selection (preference) result of the user U may be proposed. After the user U sets one or more domains in their life, information for clarifying a sense of values may be presented for each domain. For example, the following sentence may be presented in order to have the user U input a description (setting of an ideal self-image) that makes the sense of values concrete.
“Regarding the domain ′ family′ chosen earlier, what kind of person would you like to become?”

The proposal to set the action goal is a proposal for the user U to set the action goal aligned with the sense of values.

The setting of the action goal aligned with the setting content (description or the like) of the sense of values described above is proposed to the user U.

For example, when past activity records of the user U are obtained as the log information of the support application 40 of the user information 30, some activities included therein are proposed as candidates of the action goal. A plurality of action goals may be set. In addition, regarding the set action goal, in order to make details concrete, such as the date and time for executing an action related to the action goal, and a method for achieving the execution, for example, the following additional questions may be presented.
“How about ‘making a list of extracurricular activities for your child available in the neighborhood’ in order to achieve ‘I want to be a good parent for my child’ that you set before?”
“When is the convenient date and time to execute the action related to the action goal?”

The proposal to correct the sense of values and the proposal to correct the action goal are proposals for the user U to correct the sense of values and the action goal set (for example, described) by the user U according to the above-described proposals. The proposals for the correction may be performed based on a comparison result between the sense of values and the action goal actually set by the user U and the sense of values and the action goal proposed by the support information 91 that can be generated by the information generation unit 9 based on the current user model 80.

For example, the information generation unit 9 calculates the degree of deviation between the sense of values and the action goal set by the user U and the sense of values and the action goal that can be proposed (estimated) based on the user model 80. The degree of deviation may be calculated using the distance between topics included in sentences indicating the senses of values and the action goals. For example, each sentence is associated with predetermined classification information. The distance between pieces of the classification information is determined in advance, and the degree of deviation between these sentences is calculated based on the distance. The larger the distance is, the larger the degree of deviation is, and the smaller the distance is, the smaller the degree of deviation is. The present disclosure is not limited thereto, and the degree of deviation may be calculated using various known methods. One example is a calculation based on cosine similarity.

The information generation unit 9 may generate support information 91 including the proposal to correct the sense of values and the action goal of the user U when the degree of deviation exceeds a set threshold value. For example, a dialogue sentence may be presented that prompts the user U to correct (reconsider) the setting of the sense of values and the action goal or that asks the user U the reason of setting the sense of values and the action goal.

By proposing correcting the sense of values and the action goal, it is possible to provide the user U with an opportunity to repeatedly reconsider the set sense of values and action goal, as in actual counseling.

The reminder is a reminder for supporting the execution of an action related to the set action goal. For example, the reminder is provided as notification at any timing before the action is executed.

In one embodiment, a situation (time of day, place, or the like) suitable for the reminder may be estimated. In the following, it is assumed that the information generation unit 9 is one that performs estimation. The information generation unit 9 estimates the situation suitable for the reminder from the obtained user information 30. Examples of the situation include the time of day, a place, and the like. A dialogue with the user U for determining (specifying) these situations may be performed in advance. When determining that the user U is in the estimated situation, the information generation unit 9 generates support information 91 including the reminder. For example, when the current time is in the time of day suitable for the reminder or the position information of the user U indicates a place suitable for the reminder, the support information 91 including the reminder is generated and presented to the user U.

A re-reminder (additional reminder) may also be made. In this case, it is determined whether or not the re-reminder is possible. This determination may be made manually or automatically.

When the determination is made manually, for example, the user U operates the user terminal 2 to determine whether or not the re-reminder is possible.

When the determination is made automatically, whether or not the re-reminder is possible may be determined on a rule basis based on an execution state of the action related to the action goal set by the user U. For example, when the user U has already executed the action related to the action goal, it is determined that the re-reminder is not possible (unnecessary). When the user U has not executed the action related to the action goal, more specifically, when the motivation of the user U for the therapy does not decrease, it is determined that the re-reminder is possible (necessary). Whether or not the motivation of the user U decreases is determined based on, for example, dialogue information or the like.

The feedback is feedback about the execution of the action related to the action goal, and examples thereof include encouragement, advice, and the like. For example, when the user U executes an action related to a certain action goal, support information 91 including words of encouragement is generated and presented. On the contrary, when the user U does not execute the action related to the action goal as scheduled, support information 91 including advice for the execution is generated and presented.

Information other than the above is illustrated as other information. An example of the other information is decoration. For example, information on expressive elements and the like, such as the appearance of a character displayed during a dialogue, the style of the dialogue, a change in color on the screen, a sound effect, and the like can be the support information 91. By using such support information 91, for example, the user U can easily input information, and the support effect can be enhanced.

Another example of the other information is adjustment. For example, a dialogue sentence for determining the time of day, frequency, and the like (night, several days a week, and the like) of dialogues between the user U and the support system 1 may be generated as the support information 91. Further, a question sentence for determining a presentation method of the support information 91 (presentation via the support application 40, presentation via an external mailer, or the like) may be generated as the support information 91.

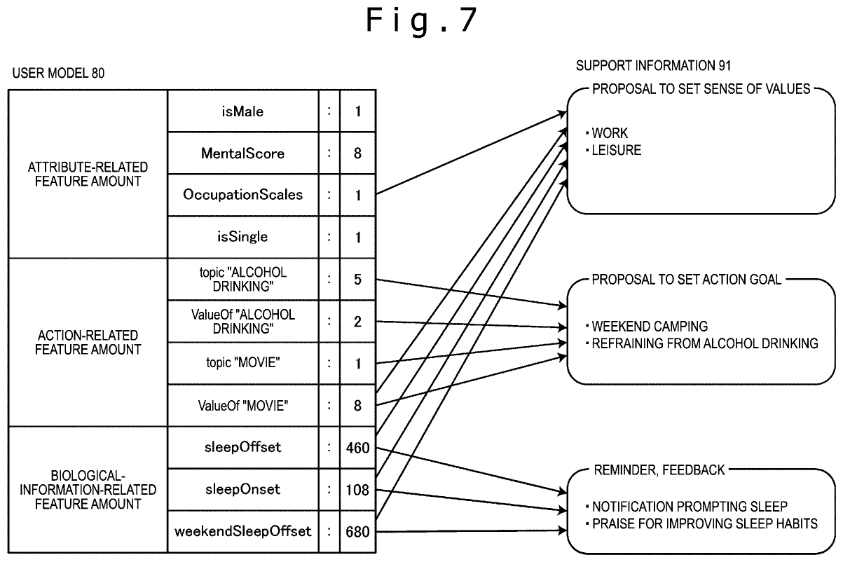

A specific example of generation of the support information 91 based on the user model 80 will be described with reference to FIG. 7.

FIG. 7 is a diagram illustrating the specific example of the generation of the support information 91 based on the user model 80. At the left part of FIG. 7, the same user model 80 as that of FIG. 4 described above is illustrated. For example, support information 91 including a proposal to set a sense of values, a proposal to set an action goal, a reminder, and feedback is generated from the user model 80, as illustrated at the right part of FIG. 7.

The proposal to set the sense of values will be described. From the occupation-related feature amount (OccupationScales) and the biological-information-related feature amounts included in the user model 80, it is understood that the user U goes to bed late, the gap between the wake-up times on weekdays of holidays is large, and the user U spends time on work on weekdays. From the feature amounts related to movie evaluation (Topic “movie” and ValueOf “movie”), it is understood that mood evaluation at the time of movie watching is high. It is also understood that there is no feature amount related to a human relationship such as a family or a friend. From these matters, “work” and “leisure” are estimated as being important among candidates of the sense of values in the life of the user U.

Information for proposing setting these matters as the sense of values is generated as the support information 91. The above-described feature amounts that serve as the basis of the proposal and related descriptions may also be included in the support information 91.

The proposal to set the action goal will be described. For example, when the user U selects “leisure” in response to the proposal to set the sense of values described above, support information 91 including a proposal to, on a weekend, watch a “movie”, which is highly evaluated, or perform a highly evaluated action (for example, camping) performed by another user U as leisure is generated in response to the selection. In addition, since the user U has a low evaluation of alcohol drinking, the time of day and the situation of the alcohol drinking are input through a dialogue, and support information 91 including a proposal to refrain from alcohol drinking at the same time of day is generated. The above-described feature amounts that serve as the basis of the proposal and related descriptions may also be included in the support information 91.

The reminder and the feedback will be described. For example, the time of day suitable for the user U to go to bed is estimated, and support information 91 including a notification for prompting the user U to sleep in that time of day is generated. In addition, support information 91 including a dialogue for praising (commending or the like) improvement of a sleep week is generated.

Referring back to FIG. 1, for example, as described above, various types of support information 91 are generated based on the user model 80 and presented to the user U.

In one embodiment, when the support information 91 is generated, a support information candidate 90 stored in the storage section 10 may be referred to. The support information candidate 90 is information that can be used as the information generation unit 9 or information that serves as the basis of such information.

In the example illustrated in FIG. 1, there are a plurality of support information candidates 90 corresponding to the plurality of user models 80 of the plurality of users U, and the support information candidates 90 are stored in the storage section 10 in association with the user models 80. That is, pairs each including the user model 80 and the corresponding support information 91 are stored in the storage section 10.

The support information candidate corresponding to the user model 80-1 is referred to as a support information candidate 90-1 and illustrated. The support information candidate corresponding to the user model 80-2 is referred to as a support information candidate 90-2 and illustrated. The support information candidate corresponding to the user model 80-3 is referred to as a support information candidate 90-3 and illustrated. When these support information candidates are not particularly distinguished from each other, each support information candidate is simply referred to as a support information candidate 90.

The information generation unit 9 generates support information 91 to be presented to the user U based on the support information candidate 90 corresponding to the user model 80 of the user U. For example, support information 91 to be presented to the user U-1 is generated based on the support information candidate 90-1. Support information 91 to be presented to the user U-2 is generated based on the support information candidate 90-2. Support information 91 to be presented to the user U-3 is generated based on the support information candidate 90-3. By referring to the support information candidate 90 individualized for each user U, it is possible to generate the support information 91 suitable for presentation to the user U.

The support information 91 may be not only prepared in advance and stored in the storage section 10, but also updated as appropriate based on the corresponding user information 30 and user model 80. For example, the support information candidate 90 may be configured to include a sense of values and an action goal proposed by the support information 91 (presented to the user U) generated by the information generation unit 9. The support information candidate 90 may be configured to include a sense of values and an action goal set by the user U. By customizing the support information candidate 90 for each user U, the possibility that more appropriate support information 91 can be generated increases.

In one embodiment, at the time of supporting the setting of a sense of values and an action goal of a certain user U, the user model 80 and the support information candidate 90 of another user U may be referred to. For example, at the time of supporting the setting of the sense of values and the action goal of the user U-1, the information generation unit 9 may refer to the user model 80-2 and the support information candidate 90-2 of the user U-2, and generate, based on these pieces of information, support information 91 including a proposal to set a sense of values and an action goal similar to those of the user U-2. For example, the support information 91 may include the following sentence.

“Other users have set such senses of values and action goals.”

In the above proposal, necessary information may be acquired from the user model 80-2, including the attributes of the user U-2 such as an age and a family structure, and presented to the user U-1.

In order to estimate a situation suitable for a reminder to execute an action related to an action goal of a certain user U, the user model 80 and the support information candidate 90 of another user U may be referred to. For example, when the user model 80-1 of the user U-1 and the user model 80-2 of the user U-2 are similar to each other, a situation similar to a situation of a reminder that has increased the action execution rate of the user U-2 may be estimated as a situation suitable for a reminder to the user U-1. Accordingly, the timing of the reminder to the user U-1 can be automatically determined.

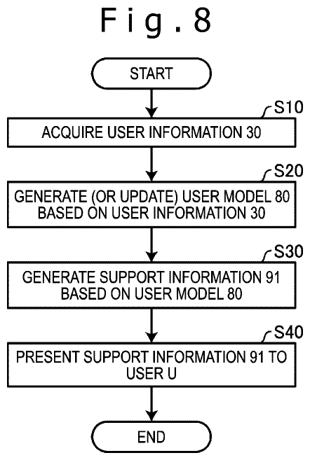

FIGS. 8 to 12 are flowcharts illustrating an example of processing (support method) executed in the support system 1. The description of the contents overlapping with the above will be omitted as appropriate.

FIG. 8 illustrates a basic processing flow. In step S10, user information 30 is acquired. The acquisition unit 3 of the user terminal 2 acquires the user information 30. The acquired user information 30 is transmitted from the user terminal 2 to the information processing apparatus 6.

In step S20, a user model 80 is generated (or updated). The model generation unit 8 of the information processing apparatus 6 generates the user model 80 based on the user information 30. When the user model 80 has already been generated, the user model 80 is updated.

In step S30, support information 91 is generated based on the user model 80. The information generation unit 9 of the information processing apparatus 6 generates the support information 91 based on the user model 80. A support information candidate 90 corresponding to the user model 80 may be referred to.

In step S40, the support information 91 is presented. The support information 91 generated in previous step S30 is transmitted from the information processing apparatus 6 to the user terminal 2. The presentation unit 5 of the user terminal 2 presents the support information 91 to the user U.

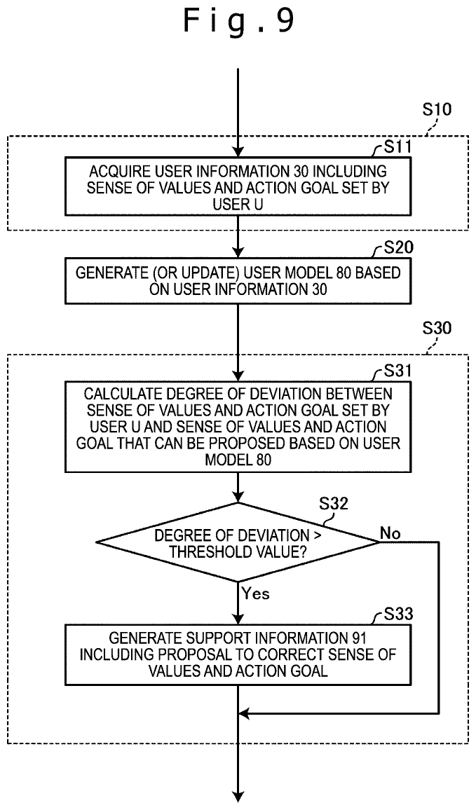

For example, as described above, the setting or the like of a sense of values and an action goal of the user U is supported. FIGS. 9 to 12 illustrate some examples of a more specific processing flow.

FIG. 9 illustrates a processing flow in the case of generating support information 91 including a proposal to correct a sense of values and an action goal. Specific processing of step S10 is illustrated as step S11. User information 30 including a sense of values and an action goal set by the user U is acquired.

The processing of step S20 is as described above, and a user model 80 is generated (or updated).

Specific processing of step S30 is illustrated as step S31 to step S33. These processing operations are executed by the information generation unit 9 of the information processing apparatus 6.

In step S31, the degree of deviation between the sense of values and the action goal set by the user U and a sense of values and an action goal that can be proposed based on the user model 80 is calculated.

In step S32, it is determined whether the degree of deviation exceeds a threshold value (surpasses the threshold value). When the degree of deviation exceeds the threshold value (Yes in step S32), the processing proceeds to step S33. Otherwise (No in step S32), the processing of step S33 is skipped.

In step S33, support information 91 including a proposal to correct the sense of values and the action goal is generated. Thereafter, the processing of step S30 is completed.

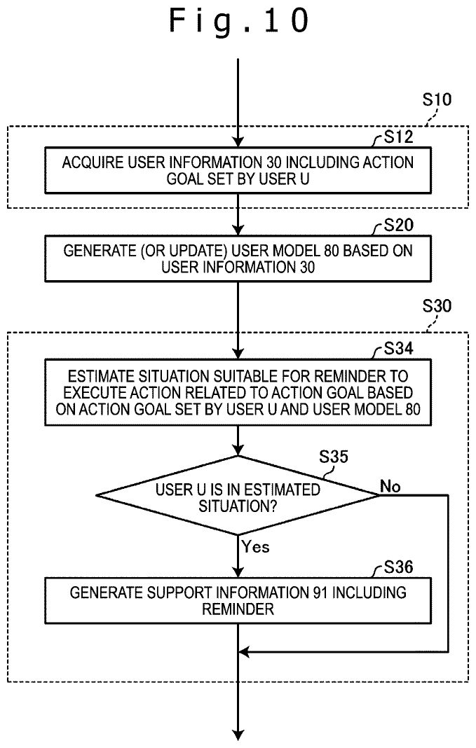

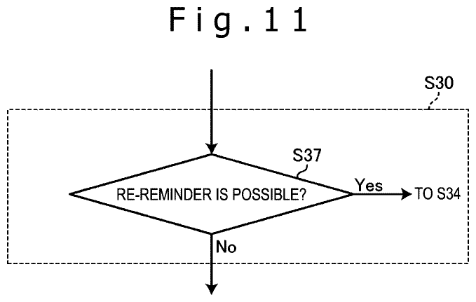

FIGS. 10 and 11 illustrate a processing flow in the case of generating support information 91 including a reminder to execute an action related to an action goal.

In FIG. 10, specific processing of step S10 is illustrated as step S12. The acquisition unit 3 of the user terminal 2 acquires user information 30 including an action goal set by the user U.

The processing of step S20 is as described above, and a user model 80 is generated (or updated).

Specific processing of step S30 is illustrated as step S34 to step S36. In step S34, the information generation unit 9 of the information processing apparatus 6 estimates, based on the action goal set by the user U and the user model 80, a situation suitable for a reminder to execute an action related to the action goal.

In step S35, the information generation unit 9 determines whether or not the user U is in the estimated situation (step S35). When the user U is in the estimated situation (Yes in step S35), the processing proceeds to step S36. Otherwise (No in step S35), the processing of step S36 is skipped.

In step S36, the information generation unit 9 generates support information 91 including the reminder. Thereafter, the processing of step S30 is completed.

In FIG. 11, specific processing of step S30 related to a re-reminder is illustrated as step S37. The information generation unit 9 of the information processing apparatus 6 determines whether or not a re-reminder is possible. When the re-reminder is possible (Yes in step S37), the processing proceeds to step S34 in FIG. 10 described above. Otherwise (No in step S37), the processing of step S30 is completed.

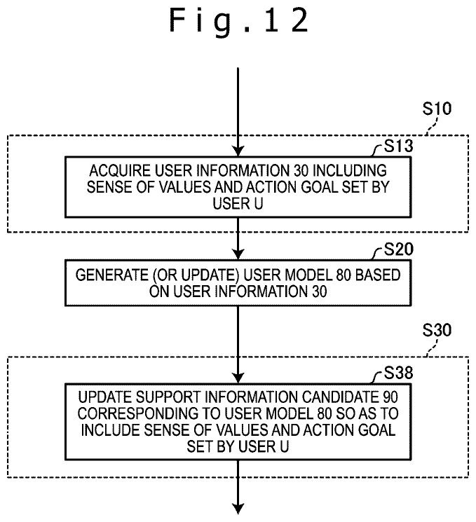

FIG. 12 illustrates a processing flow in the case of storing a pair of a user model 80 and a support information candidate 90 in the storage section 10.

Specific processing of step S10 is illustrated as step S13. The acquisition unit 3 of the user terminal 2 acquires user information 30 including a sense of values and an action goal set by the user U.

The processing of step S20 is as described above, and a user model 80 is generated (or updated).

Specific processing of step S30 is illustrated as step S38. The information generation unit 9 of the information processing apparatus 6 updates a support information candidate 90 corresponding to the user model 80 so as to include the sense of values and the action goal set by the user U. It can also be considered that a pair of the user model 80 and the updated support information candidate 90 is stored in the storage section 10. Thereafter, the processing of step S30 is completed.

In the above processing of step S38, as described above, the support information candidate 90 may be updated so as to include a sense of values and an action goal proposed by support information 91 generated by the information generation unit 9.

### 2. Modified Example

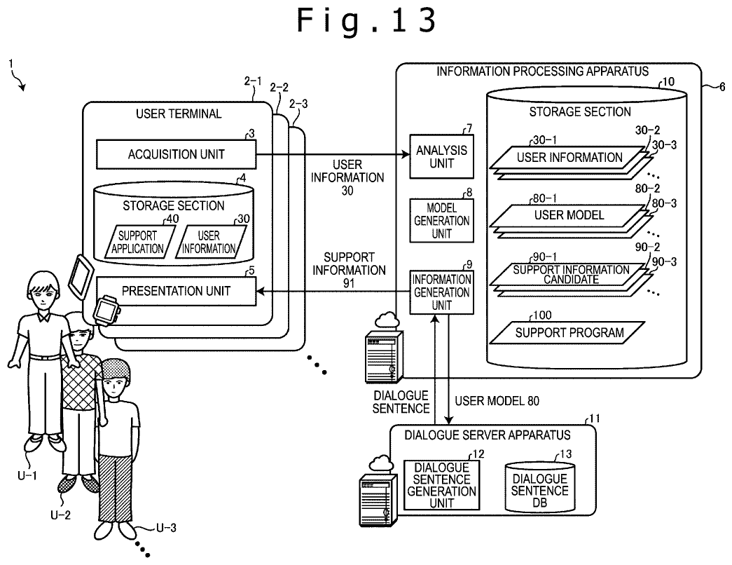

Some of the functions of the information processing apparatus 6 described above may be provided in an apparatus different from the information processing apparatus 6, for example, a server apparatus or the like provided outside the support system 1. The information processing apparatus 6 supports the user U by using the function of such an external apparatus. An example will be described with reference to FIG. 13.

FIG. 13 is a diagram illustrating a modified example. A dialogue server apparatus 11 is illustrated as the external device. The dialogue server apparatus 11 is a server apparatus specialized in generating a dialogue sentence. The information generation unit 9 of the information processing apparatus 6 generates support information 91 including a dialogue sentence between the user U and the support system 1 by using the function of the dialogue server apparatus 11.

In this example, information of a user model 80 is transmitted from the information processing apparatus 6 to the dialogue server apparatus 11. In addition, information of a dialogue sentence input by the user U (an example of user information 30) can be transmitted from the information processing apparatus 6 to the dialogue server apparatus 11.

The dialogue server apparatus 11 generates a dialogue sentence with the user U based on the information such as the user model 80 from the information processing apparatus 6. The generated dialogue sentence is transmitted from the dialogue server apparatus 11 to the information processing apparatus 6. The information generation unit 9 of the information processing apparatus 6 generates support information 91 including the dialogue sentence from the dialogue server apparatus 11.

As the functional blocks of the dialogue server apparatus 11, a dialogue sentence generation unit 12 and a dialogue sentence DB 13 are illustrated. The dialogue sentence DB 13 stores various types of information that can be used to generate a dialogue sentence. The dialogue sentence generation unit 12 generates a dialogue sentence to be presented to the user model 80 based on the user model 80 while referring to the dialogue sentence DB 13. Various known dialogue engines and the like may be used.

By also using the externally provided dialogue server apparatus 11, it is possible to additionally construct a dialogue environment in accordance with the domain of the dialogue content and the attributes of the user U. For example, the possibility that support information 91 including a dialogue sentence suitable for presentation can be generated for each user U is increased.

The support system 1 described above including that of the above modified example may be incorporated as a part of a software application implementing a series of behavior activation therapies. In this case, the input of the user U to an external system or another module of the same application may be referred to in the support system 1. Examples of the input include sociodemographic information of the user U, action record information that the user U has filled out to examine their activity pattern, a subjective evaluation for action, and the like.

### 3. Example of Hardware Configuration

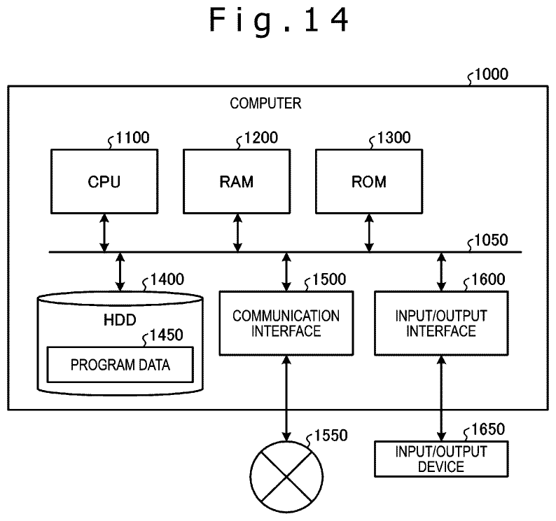

FIG. 14 is a diagram illustrating an example of a hardware configuration of an apparatus or the like. The user terminal 2 or the information processing apparatus 6 described above is implemented by, for example, a computer 1000 illustrated in FIG. 14.

The computer 1000 includes a CPU 1100, a RAM 1200, a read only memory (ROM) 1300, a hard disk drive (HDD) 1400, a communication interface 1500, and an input/output interface 1600. The units of the computer 1000 are connected to each other via a bus 1050.

The CPU 1100 operates based on programs stored in the ROM 1300 or the HDD 1400 to control the units. For example, the CPU 1100 loads the programs stored in the ROM 1300 or the HDD 1400 to the RAM 1200 and executes processing operations corresponding to various programs. Examples of the programs include the support application 40 and the support program 100 described above with reference to FIG. 1.

The ROM 1300 stores a boot program such as a basic input output system (BIOS) executed by the CPU 1100 at the time of activating the computer 1000, a program depending on the hardware of the computer 1000, and the like.

The HDD 1400 is a computer-readable recording medium that non-transitorily records the programs executed by the CPU 1100, and data and the like used by the programs. In particular, the HDD 1400 is a recording medium that records a program for the information processing method according to the present disclosure, which is an example of program data 1450.

The communication interface 1500 is an interface for the computer 1000 to connect to an external network 1550 (for example, the Internet). For example, the CPU 1100 receives data from another apparatus and transmits data generated by the CPU 1100 to the other apparatus via the communication interface 1500.

The input/output interface 1600 is an interface for connecting an input/output device 1650 and the computer 1000. For example, the CPU receives data from an input device such as a keyboard or a mouse via the input/output interface 1600. The CPU 1100 also transmits data to an output device such as a display, a speaker, or a printer via the input/output interface 1600. The input/output interface 1600 may also function as a media interface that reads a program or the like recorded on a predetermined computer-readable recording medium (media). Examples of the media include an optical recording medium such as a digital versatile disc (DVD) or a phase change rewritable disk (PD), a magneto-optical recording medium such as a magneto-optical disk (MO), a tape medium, a magnetic recording medium, a semiconductor memory, and the like.

When the computer 1000 functions as the user terminal 2 or the information processing apparatus 6 described above, the CPU 1100 of the computer 1000 executes programs loaded on the RAM 1200 to implement the functions of the acquisition unit 3 and the presentation unit 5 or the functions of the analysis unit 7, the model generation unit 8, and the information generation unit. The programs may be stored in the HDD 1400. The CPU 1100 reads the program data 1450 from the HDD 1400 and executes the program data 1450, but as another example, the CPU 1100 may acquire a program from another apparatus via the external network 1550.

Each of the above-described components may be configured using a general-purpose member, or may be configured by hardware specialized in the function of each component. Such a configuration can be appropriately changed according to the technical level at the time of implementation.

### 4. Summary

The technologies described above are specified as follows, for example. One of the disclosed technologies is the support system 1. As described with reference to FIGS. 1 to 13 and the like, the support system 1 includes the acquisition unit 3 that acquires the user information 30 of the user U, the model generation unit 8 that generates the user model 80 of the user U based on the user information 30, the information generation unit 9 that generates, based on the user model 80, the support information 91 for supporting the setting of the sense of values and the action goal of the user U, and the presentation unit 5 that presents the support information 91 to the user U.

According to the support system 1 described above, the support information 91 for supporting the setting of the sense of values and the action goal of the user U is generated based on the user model 80 of the user U. By presenting such support information 91 to the user U, it is possible to support the setting of the sense of values and the action goal of the user U.

As described with reference to FIGS. 3 to 5 and the like, the user model 80 may include the feature amount obtained from the user information 30, and the feature amount of the user model 80 may include at least one of the feature amount related to the attribute of the user U, the feature amount related to the action of the user U, or the feature amount related to the biological information of the user U (for example, the feature amount may be described using the vector). For example, by using such a user model 80 including the feature amount related to the user U, it is possible to support the setting of the sense of values and the action goal in line with the feature of the user U.

As described with reference to FIGS. 1, 3, and the like, the acquisition unit 3 may acquire the plurality of pieces of user information 30 of the plurality of users U, and the feature amount of the user model 80 may include the feature amount (comparison feature amount) related to comparison with another user U. For example, the comparison feature amount may include the feature amount ranking high among the plurality of users. This increases the number of usable feature amounts, enhances the expressiveness of the user model 80, and enables support that is more in line with the feature of the user U.

As described with reference to FIGS. 1, 2, and the like, the acquisition unit 3 may be provided at the user terminal 2 used by the user U, and the user information 30 may include at least one of the operation information of the user terminal 2, the biological information of the user U, the log information of the support application 40 that operates on the user terminal 2 and that supports the setting of the sense of values and the action goal of the user U, or the information on the dialogue performed by the user U using the user terminal 2. The log information of the support application 40 may include at least one of the demographics of the user U, the subjective scale of the user U, the progress of the therapy for the user U, the record of the therapeutic activity for the user U, or the subjective evaluation of the therapeutic activity for the user U. For example, the user model 80 can be generated based on such user information 30.

As described with reference to FIGS. 6, 7, 9 to 11, and the like, the support information 91 may include the proposal to set the sense of values of the user U and the proposal to set the action goal of the user. The support information 91 may include the proposal to correct the sense of values of the user U and the proposal to correct the action goal of the user U. The support information 91 may include the reminder to execute the action related to the action goal. The support information 91 may include the feedback about the execution of the action related to the action goal. For example, by presenting such support information 91 to the user U, it is possible to support the setting of the sense of values and the action goal of the user U.

As described with reference to FIGS. 6, 9, and the like, the information generation unit 9 may generate the support information 91 including the proposal to correct the sense of values of the user U and the proposal to correct the action goal of the user U when the degree of deviation between the sense of values and the action goal set by the user U and the sense of values and the action goal that can be proposed based on the user model 80 exceeds the threshold value. Accordingly, as in actual counseling, it is possible to provide the user U with an opportunity to repeatedly reconsider the set sense of values and action goal.

As described with reference to FIGS. 1, 12, and the like, the support system 1 may include the storage section 10 that stores a pair of the user model 80 and the corresponding support information candidate 90, and the information generation unit 9 may generate the support information 91 based on the support information candidate 90. By holding the pair of the user model 80 and the support information candidate 90 as a DB for each user U, it is possible to support the setting of the sense of values and the action goal of the user U with reference to the user model 80 and the support information candidate 90 of another user U, for example.

As described with reference to FIGS. 1, 6, 12, and the like, the support information 91 may include the proposal to set the sense of values of the user U and the proposal to set the action goal of the user U, and the support information candidate 90 may be updated such that the support information candidate 90 includes the sense of values and the action goal proposed by the support information 91. The support information candidate 90 may be updated such that the support information candidate 90 includes the sense of values and the action goal set by the user U. By customizing the support information candidate 90 for each user U in this manner, the possibility that more suitable support information 91 can be generated is increased.

The support method described with reference to FIGS. 1 to 13 and the like is also one of the disclosed technologies. The support method includes acquiring the user information 30 of the user U (step S10), generating the user model 80 of the user U based on the user information 30 (step S20), generating, based on the user model 80, the support information 91 for supporting the setting of the sense of values and the action goal of the user U (step S30), and presenting the support information 91 to the user U (step S40). As described above, the setting of the sense of values and the action goal of the user U can be supported by such a support method.

The information processing apparatus 6 described with reference to FIGS. 1 to 13 and the like is also one of the disclosed technologies. The information processing apparatus 6 includes the model generation unit 8 that generates, based on the user information 30 of the user U, the user model 80 of the user U, and the information generation unit 9 that generates, based on the user model 80, the support information 91 for supporting the setting of the sense of values and the action goal of the user U. As described above, such an information processing apparatus 6 can also support the setting of the sense of values and the action goal of the user U.

Note that the effects described in the present disclosure are mere examples, and are not limited to the disclosed contents. The present disclosure may have other effects.

Although the embodiment of the present disclosure has been described above, the technical scope of the present disclosure is not limited to the above-described embodiment alone, and various modifications can be made without departing from the gist of the present disclosure. Further, the constituent elements of different embodiments and modified examples may be combined as appropriate.

Note that the present technology can also have the following configurations.
(1)

A support system including
an acquisition unit configured to acquire user information of a user,
a model generation unit configured to generate a user model of the user based on the user information,
an information generation unit configured to generate, based on the user model, support information supporting setting of a sense of values and an action goal of the user, and
a presentation unit configured to present the support information to the user.
(2)

The support system according to (1), wherein
the user model includes a feature amount obtained from the user information, and
the feature amount of the user model includes at least one of
a feature amount related to an attribute of the user,
a feature amount related to an action of the user, or
a feature amount related to biological information of the user.
(3)

The support system according to (2), wherein
in the user model, the feature amount is described using a vector.
(4)

The support system according to (2) or (3), wherein
the acquisition unit acquires a plurality of pieces of the user information of a plurality of the users, and
the feature amount of the user model includes a feature amount related to comparison with another user of the plurality of the users.
(5)

The support system according to (4), wherein
the feature amount related to the comparison includes a feature amount ranking high among the plurality of the users.
(6)

The support system according to any of (1) to (5), wherein
the acquisition unit is provided at a user terminal used by the user,
the user information includes at least one of
operation information of the user terminal,
biological information of the user,
log information of a support application, the support application being configured to operate on the user terminal and support the setting of the sense of values and the action goal of the user, or
information on a dialogue performed by the user using the user terminal.
(7)

The support system according to (6), wherein
the log information of the support application includes at least one of
demographics of the user,
a subjective scale of the user,
progress of a therapy for the user,
a record of a therapeutic activity for the user, or
a subjective evaluation of the therapeutic activity for the user.
(8)

The support system according to any of (1) to (7), wherein
the support information includes a proposal to set the sense of values of the user and a proposal to set the action goal of the user.
(9)

The support system according to any of (1) to (8), wherein
the support information includes a proposal to correct the sense of values of the user and a proposal to correct the action goal of the user.
(10)

The support system according to (9), wherein
the information generation unit generates the support information including the proposal to correct the sense of values of the user and the proposal to correct the action goal of the user when a degree of deviation between the sense of values and the action goal set by the user and the sense of values and the action goal proposable based on the user model exceeds a threshold value.
(11)

The support system according to any of (1) to (10), wherein
the support information includes a reminder to execute an action related to the action goal.
(12)

The support system according to any of (1) to (11), wherein
the support information includes feedback about execution of an action related to the action goal.
(13)

The support system according to any of (1) to (12), including
a storage section configured to store a pair of the user model and a corresponding support information candidate, wherein
the information generation unit generates the support information based on the support information candidate.
(14)

The support system according to (13), wherein
the support information includes a proposal to set the sense of values of the user and a proposal to set the action goal of the user, and
the support information candidate is updated so as to include the sense of values and the action goal proposed by the support information.
(15)

The support system according to (13), wherein
the support information candidate is updated so as to include the sense of values and the action goal set by the user.
(16)

A support method including
acquiring user information of a user,
generating a user model of the user based on the user information,
generating, based on the user model, support information supporting setting of a sense of values and an action goal of the user, and
presenting the support information to the user.
(17)

An information processing apparatus including
a model generation unit configured to generate, based on user information of a user, a user model of the user, and
an information generation unit configured to generate, based on the user model, support information supporting setting of a sense of values and an action goal of the user.

### REFERENCE SIGNS LIST

1 Support system
2 User terminal
3 Acquisition unit
30 User information
4 Storage section
40 Support application
5 Presentation unit
6 Information processing apparatus
7 Analysis unit
8 Model generation unit
80 User model
9 Information generation unit
90 Support information candidate
91 Support information
10 Storage section
100 Support program
11 Dialogue server apparatus
12 Dialogue sentence generation unit
13 Dialogue sentence DB
U User

1. A support system comprising:
an acquisition unit configured to acquire user information of a user;
a model generation unit configured to generate a user model of the user based on the user information;
an information generation unit configured to generate, based on the user model, support information supporting setting of a sense of values and an action goal of the user; and
a presentation unit configured to present the support information to the user.

2. The support system according to claim 1, wherein
the user model includes a feature amount obtained from the user information, and
the feature amount of the user model includes at least one of
a feature amount related to an attribute of the user,
a feature amount related to an action of the user, or
a feature amount related to biological information of the user.

3. The support system according to claim 2, wherein
in the user model, the feature amount is described using a vector.

4. The support system according to claim 2, wherein
the acquisition unit acquires a plurality of pieces of the user information of a plurality of the users, and
the feature amount of the user model includes a feature amount related to comparison with another user of the plurality of the users.

5. The support system according to claim 4, wherein
the feature amount related to the comparison includes a feature amount ranking high among the plurality of the users.

6. The support system according to claim 1, wherein
the acquisition unit is provided at a user terminal used by the user,
the user information includes at least one of
operation information of the user terminal,
biological information of the user,
log information of a support application, the support application being configured to operate on the user terminal and support the setting of the sense of values and the action goal of the user, or
information on a dialogue performed by the user using the user terminal.

7. The support system according to claim 6, wherein
the log information of the support application includes at least one of
demographics of the user,
a subjective scale of the user,
progress of a therapy for the user,
a record of a therapeutic activity for the user, or
a subjective evaluation of the therapeutic activity for the user.

8. The support system according to claim 1, wherein
the support information includes a proposal to set the sense of values of the user and a proposal to set the action goal of the user.

9. The support system according to claim 1, wherein
the support information includes a proposal to correct the sense of values of the user and a proposal to correct the action goal of the user.

10. The support system according to claim 9, wherein
the information generation unit generates the support information including the proposal to correct the sense of values of the user and the proposal to correct the action goal of the user when a degree of deviation between the sense of values and the action goal set by the user and the sense of values and the action goal proposable based on the user model exceeds a threshold value.

11. The support system according to claim 1, wherein
the support information includes a reminder to execute an action related to the action goal.

12. The support system according to claim 1, wherein
the support information includes feedback about execution of an action related to the action goal.

13. The support system according to claim 1, comprising:
a storage section configured to store a pair of the user model and a corresponding support information candidate, wherein
the information generation unit generates the support information based on the support information candidate.

14. The support system according to claim 13, wherein
the support information includes a proposal to set the sense of values of the user and a proposal to set the action goal of the user, and
the support information candidate is updated so as to include the sense of values and the action goal proposed by the support information.

15. The support system according to claim 13, wherein
the support information candidate is updated so as to include the sense of values and the action goal set by the user.

16. A support method comprising:
acquiring user information of a user;
generating a user model of the user based on the user information;
generating, based on the user model, support information supporting setting of a sense of values and an action goal of the user; and
presenting the support information to the user.

17. An information processing apparatus comprising:
a model generation unit configured to generate, based on user information of a user, a user model of the user; and
an information generation unit configured to generate, based on the user model, support information supporting setting of a sense of values and an action goal of the user.
